(19) United States
(12) Patent Application Publication
(10) Pub. No.: US 2013/0029410 A1
(43) Pub. Date: Jan. 31, 2013

(54) DIGESTER TANK FOR A BIOGAS PLANT

(71) Applicants: Thomas Dory, Munchen (DE); Horst Unterlechner, Landsberg am Lech (DE)

(72) Inventors: Thomas Dory, Munchen (DE); Horst Unterlechner, Landsberg am Lech (DE)

(21) Appl. No.: 13/518,817

(22) PCT Filed: Dec. 20, 2010
(86) PCT No.: PCT/IB2010/055957

§ 371 Date: Oct. 11, 2012

(30) Foreign Application Priority Data
Dec. 22, 2009 (DE) 10 2009 059 262.8

(51) Int. Cl. C12M 1/107; C12M 1/02; C12M 1/00

(52) U.S. Cl. 435/300.1; 435/289.1

(57) ABSTRACT

The invention relates to a digester tank for a biogas plant. The interior of the digester tank is at least partially surrounded by media-tight, pressure-tight and thermally insulating walls. The digester tank has thermally insulating exterior wall segments. The thermally insulating exterior wall segments form a self-supporting polygonal ring.

This application is the national stage of International Application No. PCT/IB2010/055957, filed Dec. 20, 2010, which was not published in English, and claimed the benefit of priority to German Patent Application No. DE 10 2009 059 262.8 entitled “Fermenting Tank for a Biogas Plant” filed on Dec. 22, 2009. The entire contents of both International Patent Application No. PCT/IB2010/055957 and German Patent Application No. DE 10 2009 059 262.8 are hereby incorporated by reference.

### BACKGROUND

Due to limitations in mining and availability, Scandium is currently only produced in small quantities. While the element occurs in many ores, it is only present in trace amounts; there are no known, easily-extractable deposits of minerals containing high scandium content. Currently, only a few mines, located in Russia, Ukraine and China, produce scandium, and in each case it is made as a byproduct from the extraction of other elements and sold as scandium oxide.

The various embodiments disclosed relate to a digester tank for a biogas plant. The interior of the digester tank is at least partially surrounded by media-tight, pressure-tight and thermally insulating walls. The digester tank has thermally insulating exterior wall structure segments.

Such a digester tank for plants for biogas generation is known from DE 10 2006 033 763 A1. As shown in FIG. 5, the interior of a conventional digester tank is at least partially surrounded by pressure-tight and thermally insulating walls. For this purpose, at least one wall has at least one panel which is composed of an inner shell which is made of profiled sheet metal and located on the side of the wall which faces the interior of the digester tank. The insulating walls further comprise a coffer having a sheet metal jacket and an insulating body made of a thermally insulating material, which is supported by the jacket at least on its inward-facing side. The wall having at least one panel is joined to the opposite wall by at least one tie means which absorbs the interior pressure of the tank which acts on the two joined walls.

FIG. 5 is a partially cut-open perspective view of a known digester tank 2 of this type, the interior 3 of the digester tank 2 being visible. This digester tank 2 is made up from high elongated panels 38. Several panels 38 are joined to form a straight tank wall, and a complex interior structure 32 in the interior 3 supports the exterior walls 33, 34 and 35 and a fourth wall not shown in the drawing, so that the digester tank 2 with its rectangular cross-section retains its interior 3 even if the digester tank 2 is installed into soil.

Without the complex interior supporting structure 32, the known digester tank 2 would buckle and might collapse or fall apart. Another disadvantage of the known digester tank 2 is its complex interior structure with diagonal braces 40 and tie rods and compression members 41 and 42 which maintain the shape of the digester tank 2, so that cleaning operations in the prescribed maintenance intervals are not without hazards, because the maintenance staff has very little room for maneuver. A further disadvantage of the known digester tank 2 is the complex joint between the individual segments or panels 38 which form the exterior of the rectangular digester tank 2. Another disadvantage lies in the fact that, owing to the joining of the link plates 39 of the panels 38, which project into the interior 3, to the exterior walls 33, 34 and 35 of the tank, a media-tight seal by a suitable rubber-elastic sheet on the interior surfaces 11 of the exterior walls 33, 34 and 35 is virtually unobtainable. In addition, the anchorage of the diagonal braces 40 and tie rods and compression members 41 between the walls 33 and 35 is complex and cost-intensive.

It is therefore desirable to provide a digester tank which avoids the disadvantages of prior art and can be produced cost-effectively.

### SUMMARY

According to the various embodiments disclosed herein, a digester tank for a biogas plant is created. The interior of the digester tank may be at least partially surrounded by media-tight, pressure-tight and thermally insulating walls. The digester tank has thermally insulating light-weight exterior wall segments. The light-weight exterior wall segments form a self-supporting polygonal ring.

Such a digester tank for a biogas plant offers the advantage that it can be erected cheaply by joining pre-fabricated light-weight exterior wall segments to form a ring, and that it can be made media-tight. It offers the further advantage that the insulating light-weight exterior wall segments can be transported and assembled cost-effectively without requiring additional supporting structures in order to use the polygonal ring as a digester tank for a biogas plant. After this self-supporting polygonal ring of thermally insulating light-weight exterior wall segments has been assembled on a flat surface, a suitably media-tight protective sheet can be installed on the inside of the light-weight segments and the bottom of the digester tank, whereupon fermenting substances can be introduced and biogas which collects under a gas-tight cover on the top side of the digester tank can be obtained.

A further advantage of this digester tank lies in the fact that there is no need for any internal structure in the interior of the digester tank, so that the digester tank can be maintained, cleaned and inspected more cost effectively than the digester tank of prior art as described above, and access to the inner surfaces of the polygonal ring of light-weight exterior wall segments is made easy.

### BRIEF DESCRIPTION OF THE DRAWINGS

The accompanying drawings are presented to aid in the description of aspects of the disclosure and are provided solely for illustration of the aspects and not limitation thereof.

FIG. 1 is a diagrammatic layout of an embodiment of a digester tank according to the invention;

FIG. 2 is a diagrammatic, partially cut perspective view of the digester tank according to FIG. 1;

FIG. 3 is a diagrammatic top view of the digester tank according to FIG. 1;

FIG. 4 is a diagrammatic side view of the digester tank according to FIG. 1; and

FIG. 5 is a diagrammatic, partially cut perspective view of a digester tank from prior art.

### DETAILED DESCRIPTION

The various aspects will be described in detail with reference to the accompanying drawings. Wherever possible, the same reference numbers will be used throughout the drawings to refer to the same or like parts. References made to particular examples and implementations are for illustrative purposes, and are not intended to limit the scope of the disclosure or the claims. Alternate aspects may be devised without departing from the scope of the disclosure. Additionally, well-known elements of the disclosure will not be described in detail or will be omitted so as not to obscure the relevant details of the disclosure.

The words “exemplary” and/or “example” are used herein to mean “serving as an example, instance, or illustration.” Any aspect described herein as “exemplary” and/or “example” is not necessarily to be construed as preferred or advantageous over other aspects.

In one embodiment of the invention, the light-weight exterior wall segments may be provided with heat-insulating boards having at least one exterior cover layer of corrosion-protected sheet steel. This cover layer of corrosion-protected sheet steel may advantageously give the self-supporting polygonal ring a dimensional stability, while the heat-insulating boards with a thickness of more than 50 mm may maintain the reaction heat of the digester tank.

The heat-insulating boards may advantageously consist of closed-cell polyurethane which, owing to the closed-cell character of the polyurethane foam, cannot absorb lots of water or other fluids like a sponge and therefore retains its heat-insulating properties even if not protected against the environment. Instead of closed-cell polyurethane foam, expanded polystyrene may also be used, buts its mechanical strength and dimensional stability may not be as good as those of the polyurethane foam. In addition, polyurethane foam may be shaped to form heat-insulating boards in suitable prepared cavity dies without requiring any pressure moulds and/or cutting tools as in the case of polystyrene.

It is further provided that the corners of the polygonal ring may comprise vertically arranged groove-to-tongue transitions of the heat-insulating boards. These groove-to-tongue transitions may be made of the material of the heat-insulating boards which provide better heat insulation than prior art, in particular because the corner structures of the latter are implemented by profiled sheet metal, which increases costs on the one hand and provides a heat sink for the biomass reacting in the digester tank on the other hand.

Any remaining vertical gaps of the groove-to-tongue transitions of the heat-insulating boards at the corners of the polygonal ring may be filled with polyurethane foam within a short time and made media-tight by means of cover plates and an intermediate silicone layer once the light-weight exterior wall segments have been erected to form a ring.

In a further embodiment of the invention, the self-supporting polygonal ring may be embedded into the soil to a minimum depth relative to a site surface. In addition, the light-weight exterior wall segments may be covered by a drainage membrane and by a sealing membrane made of a non-woven fabric against draining water and fermenting substances, a burl foil being secured to the non-woven fabric. This burl foil offers the advantage of improved water and fluid discharge, so that water, moisture and fluid can be directed via the drainage membrane to a drainage line arranged around the self-supporting polygonal ring and introduced, with the aid of the drainage line, into a drainage ditch of the biogas plant. Any biomass separated in the drainage ditch can be returned to the digester tank.

Advantageously, a minimum excavation of 2.5 m may be provided for embedding the polygonal ring of the digester tank into the soil. The external dimensions of the light-weight exterior wall segments in terms of their width b_{A} of several 10 cm and their height h_{A} of several meters as well as their thickness d_{A} of several centimeters are chosen accordingly for the implementation of such a polygonal external ring.

In one embodiment of the invention, these light-weight exterior wall segments may be placed as a self-supporting polygonal ring on a base plate below the site surface; first, a base layer of non-reinforced concrete may be placed in a suitable excavation, followed by an at least 25 cm thick layer of reinforced concrete to serve as a base plate. A self-supporting polygonal ring erected on such a base plate may be provided, for the media-tight accommodation of a biomass, with a several millimeters thick EPDM sheet (ethylene-propylene-diene rubber sheet) which covers the interior surface of the light-weight exterior wall segments and, lying on the base plate, provides a media-tight cover for the latter. The EPDM sheet may be joined to the interior surfaces of the light-weight exterior wall segments by adhesive force by means of an adhesive layer.

This 3 mm to 5 mm thick rubber sheet of a terpolymer elastomer may be highly resistant to weather and moisture as well as to ozone and has a high thermal stability. Owing to its high elasticity and good chemical resistance, it may be used as a surface seal and therefore as a lining in one embodiment of the digester tank. A particularly advantageous property of such an EPDM sealing sheet may be its resistance to frost, in particular as is remains highly flexible in cold conditions and may be UV-resistant, therefore having a long service life. Openings in the EPDM sheet for fastening means may be sealed by means of flanged caps. Pipeline openings may additionally be secured against slipping by means of clamping rings on the pipes and supports.

In a further embodiment of the invention, the digester tank may include wall-to-ceiling transitions in which stainless steel brackets may be arranged at preset intervals. These stainless steel brackets join the ceiling of the tank to the wall, and the EPDM sheet may be held between the bracket and the wall. For this purpose, the EPDM sheet may first be secured by means of an adhesive and then secured to the interior cover layer of corrosion-protected sheet steel by means of stainless steel screws inserted through the EPDM sheet. In order to provide a media-tight seal for the legs of such a bracket against the EPDM sheet, a silicone cord may preferably be placed on the legs of the brackets before bolting, so that a silicone layer forms in the bolting process. Such brackets for the wall-to-ceiling joint may be preferably provided at intervals of several 10 cm.

In a further embodiment of the invention, a polygonal annular partition made up from partition segments may be provided within the polygonal ring of light-weight exterior wall segments. The partition segments may have a width b_{T} of several 10 cm, a height h_{T} of several meters corresponding to the height h_{A} of the light-weight exterior wall segments and a thickness d_{T} corresponding to the thickness of a stainless steel plate. As this partition may be placed in the biomass, there is no need for thermal insulation. In order to use the partition as a stabilising support element, however, the stainless steel plate of the partition may be provided with beading, edge profiles and corner bracing.

The distance between the partition and the exterior wall may be more than one metre, providing a first digester chamber with a disc-shaped layout which, owing to this distance, is easily accessible to maintenance and inspection staff

Being reinforced by the stainless steel plates, the partition may fulfill a support function, supporting at least two horizontal girders mechanically joined to the partition along the horizontal main axes x and y, which lie on the partition and extend to the self-supporting polygonal ring. These girders may at the same time support a roof structure of the digester tank.

The partition divides the interior of the digester tank into a disc-shaped first digester chamber between the self-supporting polygonal ring and the polygonal annular partition and a circular second digester chamber within the partition.

The partition segments of stainless sheet steel are perforated towards the bottom of the digester tank and gas-tight above a minimum fermenting substance level of the digester tank. In place of a perforation in the bottom region of the partition segments, a majority of the partition segments may be provided with a cut-out in the bottom region. As a result of this perforation or cut-out in the bottom region of the partition segments, forced convection may be obtained between the biomass in the first digester chamber and the biomass in the second digester chamber, for example if the biogas generated in the first digester chamber applies a gas pressure to the biomass as a result of a closed cover of the first digester chamber.

In a further embodiment of the invention, every other partition segment may have a cut-out in the bottom region, which extends across the entire width of the partition segment and has a height of several 10 cm. A height of approximately 400 mm has proved useful in ensuring the above-mentioned biomass exchange or a convention of biomass between the two digester chambers.

In a further embodiment of the invention, the partition segments may be attached to the several millimeters thick media-tight EPDM sheet on the bottom of the digester tank. Fastening means may project through the media-tight EPDM sheet for joining the partition segments to the solid base plate. In order to seal the media-tight EPDM sheet lying on the base plate, the fastening means may be provided with EPDM caps at the base. In addition, the partition segments may be provided with jointing sleeves for PVC lines, through which hot water, for example, may be introduced for heating the biomass, the lines being created with the aid of the sleeves while the partition segments are assembled into a polygonal partition.

The first digester chamber may be further provided with a media-tight seal by a disc-shaped segmented cover. This cover may also provide a media-tight seal for the circular second digester chamber if the light-weight external wall segments have the same height as the partition segments. Such sealing segments required for covering the digester chambers may be constructed in the same way as the light-weight external wall segments, each being provided with a heat-insulating board with zinc-coated or painted sheet steel and externally with a plastic panel. In an optimum cover arrangement by means of sealing segments, however, the inside may have a cover layer of stainless steel and the top will correspond to the light-weight external wall segments, which have a high thermal insulation capacity due to the heat-insulating boards. For this purpose, the top of the digester tank may be provided with girders arranged on the main axes; these support the ceiling segments which are secured thereto. It is further provided that the longitudinal joints between the ceiling segments may have sheet metal covers laid in silicone, so that these longitudinal joints retain a media-tight seal.

In a further embodiment of the invention, the partition may support a support rack for a gas reservoir, the gas reservoir being held in position between the support rack, which projects beyond the cover. Such a support rack may, however, alternatively be supported by the cover of the digester tank and joined to the partition at individual points only. In addition to the support rack for a gas reservoir, a reinforcing frame can further be provided for reinforcement below the ceiling segments, to which reinforcing frame ceiling segments and other parts installed into the digester tank can be secured.

In a further embodiment of the invention, it is provided that the partition may be surrounded by four vertical buttresses of a support structure, the buttresses extending from the bottom of the digester tank to above the first disc-shaped cover and being joined to one another by horizontal crossmembers above the second digester chamber. This support structure may improve the dimensional stability of the partition on the one hand while accommodating the gas reservoir on the other hand.

The above-mentioned support rack may support a gas bag serving as a gas reservoir, which may be located on the digester tank in the region of the second digester chamber and connected thereto via an opening in the bottom of the gas reservoir. Such a gas bag may likewise be made of EPDM sheeting and have a cylindrical shape with a double-shell conical bottom. This conical bottom may have the above-mentioned central opening which projects into the digester tank and may be sealed against the environment by a media-tight seal. Any condensate forming in the gas reservoir may therefore drip back into the digester tank through this opening.

In the first digester chamber, at least one agitator may be preferably provided for keeping the biomass in continuous motion and maintaining a forced convention via the cut-outs or perforations of the partition segments within the second digester chamber. In addition, the second digester chamber may be provided with at least one thermo-gas lifter which ensures an intensive intermixing of the biomass with the generated biogas bubbles and thus supports a natural convection, so that it may be possible to do without the forced convection and thus without an additional drive, for example for an agitator, at least in the second digester chamber.

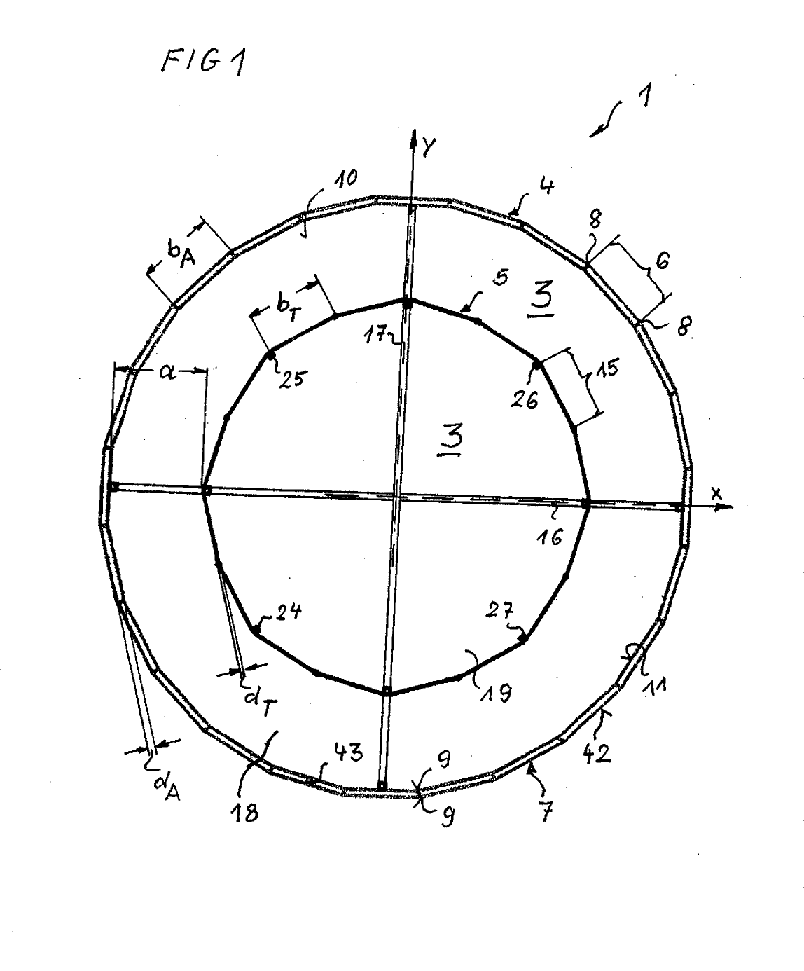

FIG. 1 is a diagrammatic layout of an embodiment of a digester tank 1. The digester tank 1 may have an interior 3 at least partially surrounded by pressure-tight, media-tight and thermally insulating walls 4. For this purpose, the digester tank 1 may be provided with thermally insulating light-weight exterior wall segments 6, the light-weight exterior wall segments 6 forming a self-supporting polygonal ring 7.

In the layout of the digester tank 1 shown in FIG. 1, the interior 3 may be divided by a partition 5 into a disc-shaped first digester chamber 18 and a second digester chamber 19. The partition 5 may be assembled into a polygonal ring 36 on a base plate 10 from a plurality of partition segments 15. The light-weight exterior wall segments 6 may have a significantly greater wall thickness d_{A} than the partition segments 15 of the polygonal partition 5 with its thickness of d_{T}, because the light-weight exterior wall segments 6 comprise heat-insulating boards made of polyurethane, while there is no need for thermal insulation in the case of the partition 5, because the partition 5 of the embodiment of the invention shown in FIGS. 1-4 do not require any heat-insulating properties.

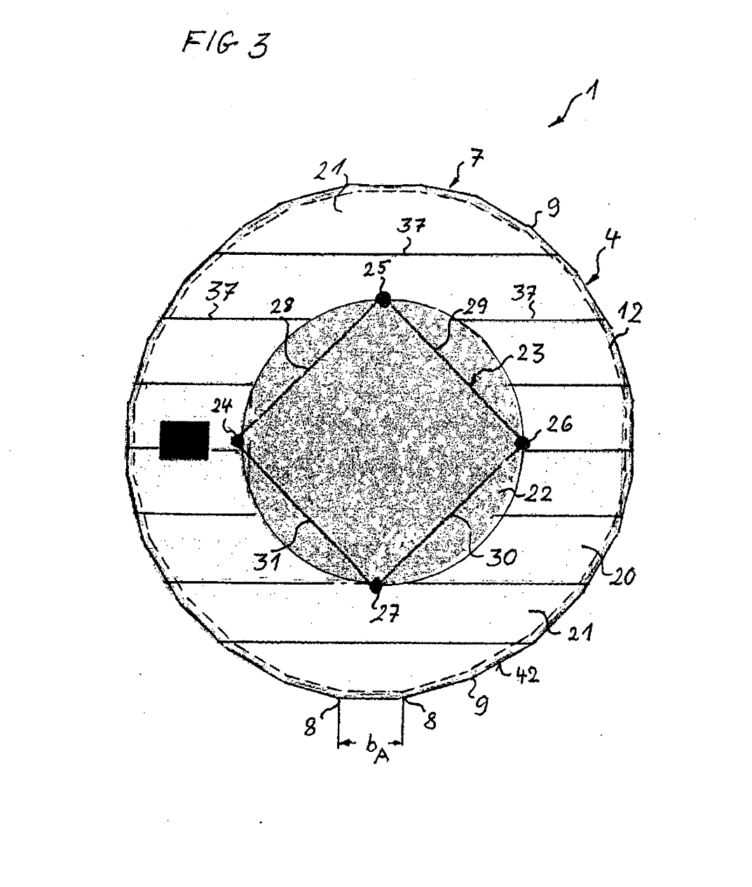

In contrast, the partition 5 may form a reinforcing and support structure for the digester tank 1 and carries the weight of a cover of the digester tank 1 shown in FIG. 3 and in addition the weight of and a support for a gas reservoir placed above the cover of the partition 5 on the digester tank 1. Such a digester tank 1 may be largely embedded into the soil, for which purpose an at least 2.5 m deep excavation may be provided and a subbase of 7 cm to 10 cm thick non-reinforced concrete may be installed after the excavation. A drainage membrane and a sealing membrane against discharging water may be placed on the base plate 10 before a layer of at least 25 cm thick reinforced concrete protected against cracking is installed, and these membranes may be secured to the outside of the light-weight external wall segments 6 after the concrete has been installed and the wall has been erected.

The light-weight external wall segments 6 may be provided with a polyurethane heat-insulating board and are joined to one another by a tongue-and-groove structure at the vertical edges of the corners 8 of the polygonal ring 7. These light-weight segments 6 may have an external cover layer 42 of zinc-coated or painted sheet steel provided with an interior heat insulating board 43 with a thickness of at least 100 mm.

The vertical joints 9 at the corners of the polygonal ring 7 may be filled with polyurethane foam and sealed by means of cover plates with a silicone intermediate layer. To protect the outer shell, the parts of the segments 6 which are covered by soil may be covered with felt, and for better water discharge a burl foil may be secured to the felt. The discharging water may be caught in a drainage line surrounding the digester tank 1 and directed to a drainage ditch of the biogas plant.

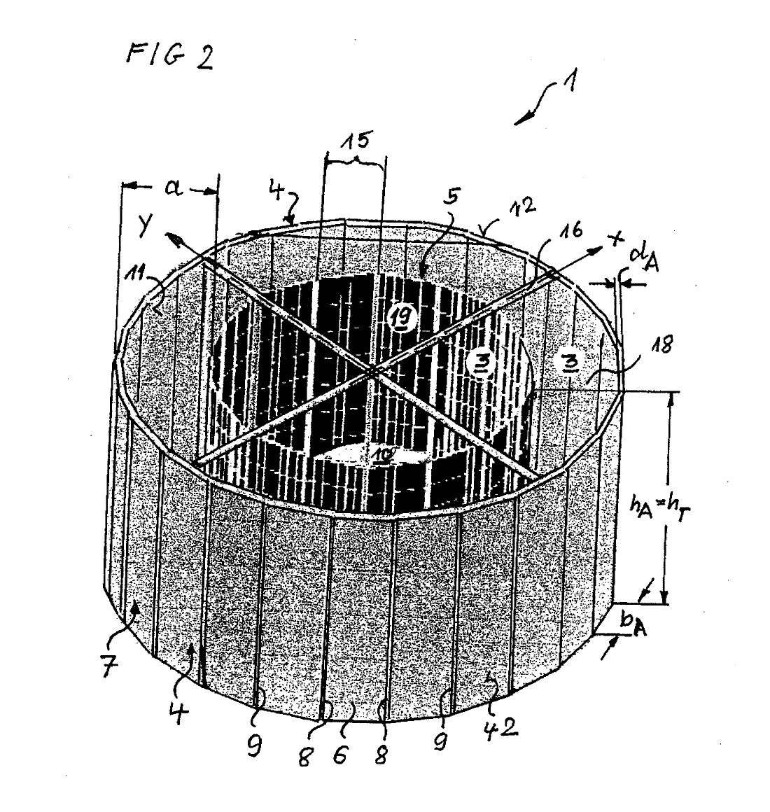

FIG. 2 is a diagrammatic, partially cut perspective view of the digester tank 1 according to FIG. 1. The partially cut perspective view shows clearly that the interior 3 within the polygonal external wall 4 may not comprise any support elements, spacers, tie rods, compression members or the like, making such a digester tank accessible for maintenance, repair and inspection. In this embodiment, a distance a may be provided between the external wall 4 and the polygonal partition 5. The height h_{A} of the light-weight external wall segments 6 may be equal to the height h_{T} of the internal partition 5. This offers the advantage that continuous ceiling segments may be placed on the wall segments for covering the digester tank 1. To support and carry such a cover as shown in FIG. 3, girders 16 and 17 additionally supported by vertical buttresses not shown in this figure are placed on the annular wall structures in the main axes x and y.

Before the interior partition 5 is installed, the digester tank 1 may be provided with a lining of EPDM sheeting on the bottom and at the interior surface 11 of the light-weight external wall segments 6; this may be secured at a wall-to-ceiling transition by a bracket which also secures ceiling segments. As an assembly aid and for additional sealing, this EPDM sheet may be secured the interior surfaces 11 of the light-weight external wall segments 6 by means of a suitable adhesive. For this purpose, the EPDM sheet of the lining may have a thickness between 3 mm and 5 mm and is, owing to its above-mentioned properties, particularly suitable for a digester tank 1. Openings in the sheet, which may be provided, for example, for attaching the partition segments 15 to the base plate 10 covered by the lining, are additionally sealed by means of flanged caps and/or EPDM caps. Openings for hot water lines and for the feed and discharge of biomass to/from the digester tank may also be mechanically secured against slipping by clamping rings on the pipes and supports.

In this embodiment, the partition segments may be 1.15 m wide and may have a distance of 1.5 m from the polygonal external ring 7. Above a lowest level for the digester tank 1, the partition segments 15 may be gas-tight, and below this level the partition segments 15 may have cut-outs with a height of approximately 400 mm in the bottom region, every other partition segment 15 in this embodiment of the invention having such a cut-out for its entire width.

In the main axes x and y, a reinforcing frame may be installed in the region of the partition 5. This reinforcing frame on the one hand reinforces the structure and on the other hand may be used to secure installed parts such as a thermo-gas lifter, an agitator as well as the ceiling segments of the cover. Vertical segment reinforcements stabilise the partition segments 15. These partition segments may be secured to the bottom by suitable fastening means such as dowels and screws and in addition sealed by means of EPDM caps. Connecting sleeves for connecting lines, for example for biomass feed and discharge, and for heating lines may be provided in some of the segments 6 and/or 15. All such lines with the exception of sludge discharge lines may pass through the light-weight external wall segments 6 below the minimum level. As far as bushings in the partition 5 between the first digester chamber 18 and the second digester chamber 19 are concerned, these may be established by means of welded screw connections. The above-mentioned lines may be made of a plastic material such as PVC (polyvinyl chloride) or of stainless steel.

FIG. 3 is a diagrammatic top view of the digester tank 1 according to FIG. 1. This top view essentially shows the structure of the ceiling, which may be composed of ceiling segments 21. These ceiling segments 21 may have the same structure as the light-weight external wall segments and may optionally be provided with a stainless steel cover on the inside and a plastic material on the outside. These ceiling segments 21 may be placed on the wall segments and secured thereto. They may be supported and secured against lifting off by the above-mentioned girders extending in the main axes x and y. The longitudinal gaps 37 between the tongue-and-groove joints may be provided with a media-tight seal of sheet metal covers laid in silicone on both sides.

The wall-to-ceiling joint 12 may be implemented by means of stainless steel brackets. The brackets may be secured by stainless steel screws. Prior to assembly, the stainless steel brackets may be bonded to the EPDM lining sheet of the digester tank 1 by means of a suitable adhesive and then may be bolted to the light-weight external wall segments. In this embodiment of the invention, the distance between these mechanical attachment points provided by stainless steel brackets may be, for example, 30 cm. The transitional gap between the wall and ceiling segments is filled with polyurethane foam, and on the outside a sheet metal bracket may be fitted for covering the ceiling segments 21 and as an additional support for the gap filled with PU foam. When this external annular and horizontal cover bracket is fitted, a silicone cord may be placed on both legs of the bracket in order to improve its weather resistance and its sealing action. The horizontal cover bracket between the wall and ceiling segments is mechanically secured by stainless steel screws.

FIG. 3 further shows the position and size of the gas bag mounted as a gas reservoir 22 on the cover of the digester tank 1 in a region of the internal partition. This gas reservoir 22 may be held by a support rack 23 of aluminium sections between vertical buttresses 24 to 27 joined to one another by horizontal crossmembers 28 to 31 as shown in FIG. 3.

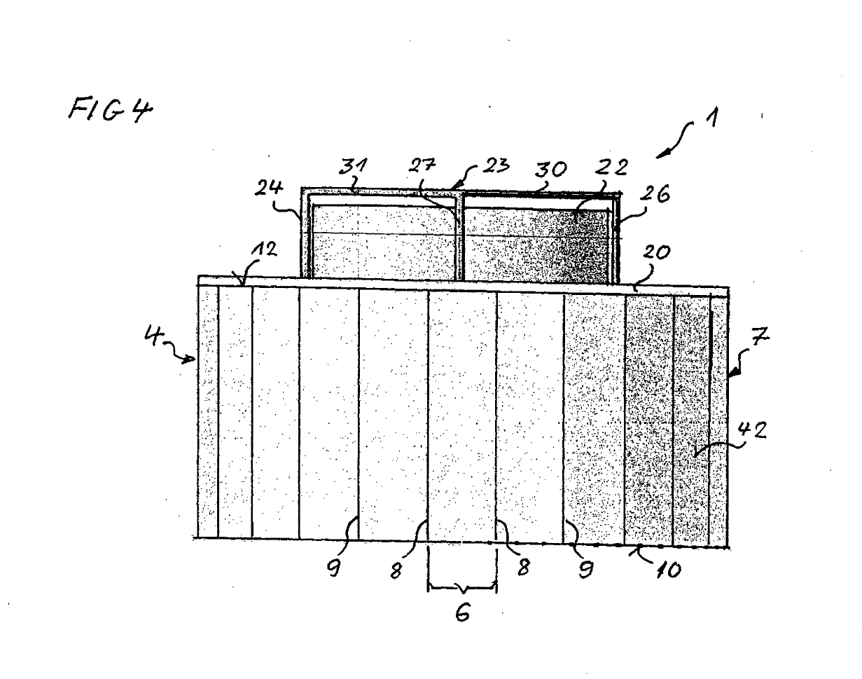

FIG. 4 is a diagrammatic side view of the digester tank 1 according to FIG. 1. This perspective representation is an overall view of the digester tank 1 assembled from a polygonal ring 7 with light-weight external wall segments 6 and ceiling segments of a cover 20 as well as the gas reservoir 22 in the region of the inner partition. This gas reservoir 22 may be made of EPDM sheeting delivered from the factory in the size and shape matching the digester tank 1 in question. In this embodiment of the invention, the gas reservoir 22 may have a volume of 25 m³ and a cylindrical shape. The bottom of the gas reservoir 22, which is not shown in the figure, may be conical and double-shelled and has an integral plastic cone which prevents the accumulation of water at the bottom of the gas reservoir, letting the water drip out through an opening in the bottom towards the digester tank.

This opening in the bottom of the gas reservoir 22 may be provided with a media-tight seal against the environment by a gas-tight joint between the gas reservoir 22 and the ceiling segments. In this embodiment of the invention, the height of the gas reservoir may be 1000 mm, and it may be divided by continuous cross-beading. Loops and eyelets for stabilising and securing a support rack 23 may be fitted to these beading elements. In this embodiment of the invention, the support rack 23 of the gas reservoir 22 may be made of light-weight aluminium sections. These light-weight aluminium sections may be secured to the cover 20 of the digester tank 1.

The wind forces may be diverted to the support structure in the digester tank 1. For protection against weathering effects, a trapezoidal sheet metal roof not shown in the drawing may be provided to avoid damage. For this purpose, the trapezoidal sheet metal roof may have a slight inclination in saddleback form. A gas line may be connected to the gas reservoir by means of sheet penetration and routed to the consumers. Above the gas reservoir, a pressure sensor may be installed for monitoring the gas pressure in the reservoir. In addition, the EPDM sheet may have a bull's eye for visual inspection.

In place of a gas reservoir and a cover provided in this embodiment of the invention, a pot-type covering hood may be placed above the digester tank 1 with its polygonal exterior wall 4 and immersed into a surge chamber. Such a surge chamber design offers the advantage that the volume of the gas reservoir adapts to the biogas pressure by raising and lowering the covering hood in the surge chamber. In this context, the covering hood may advantageously be made of EPDM sheeting. A further alternative embodiment provides that the two-chamber system may be maintained and only the first chamber may be made gas-tight with ceiling segments, while the second chamber may have a full-surface cut-out for gas discharge. A pot-type covering hood made of EPDM sheeting may then placed over the entire digester tank and surrounded by a surge chamber which makes the pot-type covering hood gas-tight against the environment.

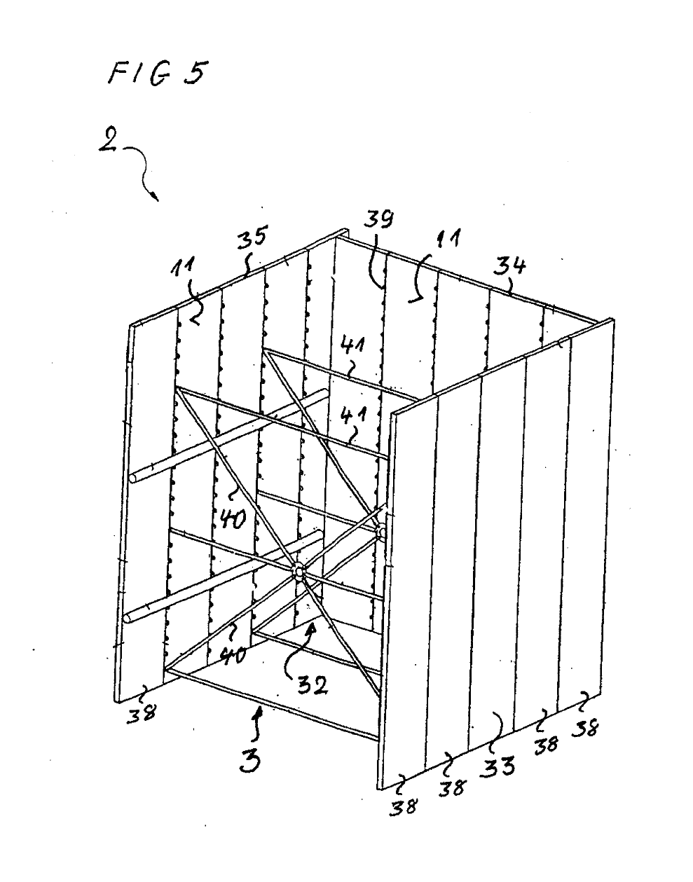

FIG. 5 is a diagrammatic, partially cut perspective view of a conventional digester tank from prior art as described above, so there is no need for a repetition at this point.

The preceding description of the disclosed aspects is provided to enable any person skilled in the art to make or use the present disclosure. Various modifications to these aspects will be readily apparent to those skilled in the art, and the generic principles defined herein may be applied to other aspects without departing from the spirit or scope of the disclosure. Thus, the present disclosure is not intended to be limited to the aspects shown herein but is to be accorded the widest scope consistent with the following claims and the principles and novel features disclosed herein.

### LIST OF REFERENCE NUMBERS

1—Digester tank (embodiment)

2—Digester tank (prior art)

3—Interior

4—Wall

5—Partition

6—Light-weight exterior wall segment

7—Self-supporting polygonal ring

8—Corners of polygonal ring

9—Vertical gap

10—Base plate

11—Interior surface of light-weight segment

12—Wall-to-ceiling transition

15—Partition segment

16—Girder (x-direction)

17 Girder (y-direction)

18—First digester chamber

19—Second digester chamber

20—Cover

21—Ceiling segment

22—Ggas reservoir

23—Support rack

24—Buttress

25—Buttress

26—Buttress

27—Buttress

28—Crossmember

29—Crossmember

30—Crossmember

31 —Crossmember

32—Interior Structure

33—Exterior Wall

34—Exterior Wall

35—Exterior Wall

36—Polygonal Ring

37—Longitudinal Gaps

38—Panel

39—Link Plate

40—Diagonal Brace

41—Tie rod and compression member

42—External cover layer

43—Heat insulating board

a Distance between exterior wall and partition

b_{A} Width of light-weight exterior wall segments

h_{A} Height of light-weight exterior wall segments

d_{A} Thickness of light-weight exterior wall segments

b_{T} Width of partition segments

h_{T} Height of partition segments

d_{T} Thickness of partition segments

x Horizontal main axis

y Horizontal main axis

1-41. (canceled)

42. A digester tank for a biogas plant, comprising:
media-tight, pressure-tight and thermally insulating walls;
an interior which is at least partially surrounded by the media-tight, pressure-tight and thermally insulating walls; and
thermally insulating light-weight exterior wall segments which form a self-supporting polygonal ring.

43. The digester tank according to claim 42, wherein the light-weight exterior wall segments are provided with heat insulating boards having at least one external cover layer of corrosion-protected sheet steel.

44. The digester tank according to claim 43, wherein the heat-insulating boards comprise a closed-cell plastic material.

45. The digester tank according to claim 44, wherein the closed-cell plastic material is polyurethane.

46. The digester tank according to claim 42, wherein corners of the polygonal ring comprise vertically arranged groove-to-tongue transitions of the thermally insulating light-weight exterior wall segments.

47. The digester tank according to claim 46, wherein vertical gaps of the groove-to-tongue transitions of the thermally insulating light-weight exterior wall segments at the corners of the polygonal ring are filled with polyurethane foam and provided with a media-tight seal by means of cover plates and a silicone intermediate layer.

48. The digester tank according to claim 42, wherein the self-supporting polygonal ring is embedded into soil to a minimum depth relative to a site surface, and wherein the thermally insulating light-weight exterior wall segments are covered by a drainage membrane and by a sealing membrane made of a non-woven fabric, and a burl foil secured to the non-woven fabric.

49. The digester tank according to claim 42, wherein the thermally insulating light-weight exterior wall segments are configured to have a width (b_{A}) of several 10 cm and a height (h_{A}) of several meters as well as a thickness (d_{A}) of several centimeters.

50. The digester tank according to claim 42, further comprising:
a drainage line surrounding the self-supporting polygonal ring, the drainage line taking up discharging water and directing it to a surge chamber.

51. The digester tank according to claim 42, further comprising:
a base plate positioned below the site surface, wherein the thermally insulating light-weight exterior wall segments are configured as a self-supporting polygonal ring on the base plate.

52. The digester tank according to claim 51, further comprising:
a media-tight EPDM sheet (ethylene-propylene-diene rubber sheet), configured to cover interior surfaces of the thermally insulating light-weight exterior wall segments and the base plate to provide a media-tight cover for the base plate, wherein the media-tight EPDM sheet is configured to be several millimeters thick.

53. The digester tank according to claim 52, further comprises:
an adhesive layer, wherein the EPDM sheet is joined by the adhesive layer to the interior surfaces of the thermally insulating light-weight exterior wall segments.

54. The digester tank according to claim 52, further comprises:
stainless steel brackets securing the EPDM sheet to wall-to-ceiling transitions.

55. The digester tank according to claim 52, further comprises:
flanged caps configured to seal openings in the EPDM sheet.

56. The digester tank according to claim 52, further comprises:
pipes;
supports;
pipeline openings formed in the EPDM sheet; and
clamping rings configured to prevent slipping of the pipeline openings by securing the pipeline openings to the pipes and supports.

57. The digester tank according to claim 52, further comprises:
a tank cover;
wall-to-ceiling transitions configured to join the tank cover to the thermally insulating light-weight exterior wall segments and to secure the EPDM sheet.

58. The digester tank of claim 57, wherein the wall-ceiling transitions are formed as stainless steel brackets, and wherein the stainless steel brackets are secured by means of an adhesive and then bolted using stainless steel screws inserted through the EPDM sheet and the thermally insulating light-weight exterior wall segments.

59. The digester tank according to claim 52, further comprising:
a polygonal annular partition assembled from partition segments, wherein the polygonal annular partition is placed within the polygonal ring of the thermally insulating light-weight exterior wall segments.

60. The digester tank according to claim 59, further comprising:
a stainless steel plate having beading, edge profiles and corner bracing, wherein the partition segments have a width (b_{T}) of several 10 cm, a height (h_{T}) of several meters and a thickness (d_{T}) corresponding to a thickness of the stainless steel plate.

61. The digester tank according to claim 59, wherein the distance (a) between the partition and the external wall is more than one metre.

62. The digester tank according to claim 59, further comprising:
at least two horizontal girders, wherein the partition supports the at least two horizontal girders mechanically joined to the partition along the horizontal main axes, which girders lie on the partition and extend to the self-supporting polygonal ring.

63. The digester tank according to claim 62, further comprising:
a disc-shaped first digester chamber; and
and a circular second digester chamber formed within the partition, wherein the partition divides the interior of the digester tank into the disc-shaped first digester chamber between the self-supporting polygonal ring and the polygonal annular partition and the circular second digester chamber formed within the partition.

64. The digester tank according to claim 59, wherein the partition segments comprise:
stainless sheet steel plates which are perforated towards the bottom of the digester tank and are configured to be gas-tight above a minimum fermenting substance level of the digester tank.

65. The digester tank according to claim 59, wherein a majority of the partition segments are provided with a cut-out in the bottom region.

66. The digester tank according to claim 59, wherein every other partition segment has a cut-out in the bottom region, which extends across the entire width of the partition segment and has a height of several 10 cm.

67. The digester tank according to claim 59, wherein the partition segments are attached to the several millimeters thick media-tight EPDM sheet on the bottom of the digester tank, wherein fastening means project through the media-tight EPDM sheet for joining the partition segments to the solid base plate and wherein the fastening means are sealed by means of EPDM caps at the bottom.

68. The digester tank according to claim 63, wherein the partition segments further comprise:
connecting sleeves for PVC lines.

69. The digester tank according to claim 68, further comprising:
a disc-shaped segmented cover to provide a media-tight seal to the first disc-shaped digester chamber.

70. The digester tank according to claim 68, wherein the cover also provides a media-tight seal for the circular second digester chamber.

71. The digester tank according to claim 70, further comprising:
ceiling segments configured to provide media-tight seals to the first disc-shaped digester chamber and circular second digester chamber.

72. The digester tank according to claim 71, wherein the ceiling segments are provided with a heat-insulating board of zinc-coating.

73. The digester tank according to claim 71, wherein the ceiling segments further comprise a painted sheet steel cover layer.

74. The digester tank according to claim 71, wherein the ceiling segments further comprise a stainless sheet steel disposed on an interior surface and a plastic material on an exterior surface.

75. The digester tank according to claim 74, wherein the ceiling segments further comprise a top segment formed from the same material as the thermally insulating light-weight exterior wall segments.

76. The digester tank according to claim 75, wherein the ceiling segments lie on wall segments and are supported on girders extending along the main axes.

77. The digester tank according to claim 76, wherein longitudinal gaps between the ceiling segments are provided with sheet metal covers laid in silicone.

78. The digester tank according to claim 77, further comprising:
a gas reservoir;
a support rack configured to support the gas reservoir, wherein the support rack is in turn supported by the partition, wherein the gas reservoir being held in position between the support rack, which projects beyond the cover.

79. The digester tank according to claim 78, further comprising:
a reinforcing frame provided in the horizontal axes of the digester tank, wherein the ceiling segments are secured to the reinforcing frame.

80. The digester tank according to claim 79, further comprising:
a plurality of vertical buttresses surrounding the partition, wherein the plurality of buttresses extend from the bottom to above the cover;
a plurality of horizontal crossmembers configured above circular second digester chamber and joining the plurality of buttress to one another.

81. The digester tank according to claim 80, wherein the gas reservoir comprises a gas bag, wherein the support rack supports the gas bag, which is located in the digester tank in the region of the second digester chamber and connected thereto by an opening in the bottom of the gas bag.

82. The digester tank according to claim 81, wherein the gas reservoir has a cylindrical shape and a double-shell conical bottom.

83. The digester tank according to claim 82, wherein the conical bottom has a central opening which projects into the digester tank, the opening being provided with a media-tight seal against the environment.

84. The digester tank according to claim 71, further comprising:
at least one agitator provided in the first digester chamber.

85. The digester tank according to claim 71, further comprising:
at least one thermo-gas lifter provided in the second digester chamber.
